(19) United States
(12) Patent Application Publication
(10) Pub. No.: US 2019/0351205 A1
(43) Pub. Date: Nov. 21, 2019

(54) Hydro Needle System

(71) Applicants: Richard H. Ford, Braselton, GA (US); John Frederick Allan McCahill, Toronto (CA); Paul L. Guilbaud, Largo, FL (US); Eric Dale Fuller, Cedartown, GA (US)

(72) Inventors: Richard H. Ford, Braselton, GA (US); John Frederick Allan McCahill, Toronto (CA); Paul L. Guilbaud, Largo, FL (US); Eric Dale Fuller, Cedartown, GA (US)

(21) Appl. No.: 16/414,180

(22) Filed: May 16, 2019

Related U.S. Application Data
(60) Provisional application No. 62/672,175, filed on May 16, 2018.

(51) Int. Cl. A61M 37/00; A61Q 19/00

(52) U.S. Cl. CPC A61M 37/0015; A61Q 19/00; A61B 2017/00761; A61M 2037/0007; A61M 2037/0023

(57) ABSTRACT

A hydro needle system includes a vial and an array of needles. Each needle in the array is in fluid communication with the vial so as to transport the liquid from the vial to the skin. An actuator is in communication with the array of needles and is configured to apply reciprocating linear motion to the needles so as to repeatedly cause the needles to reciprocate. A casing holds the vial, the needles and the actuator. The casing includes a skin contacting surface that supports the array of needles in a predetermined angular relationship with respect to the skin. At least a portion of the casing is transparent so that the liquid in the vial can be seen therethrough to determine how much liquid remains in the vial during use.

### CROSS-REFERENCE TO RELATED APPLICATION(S)

This application claims the benefit of U.S. Provisional Patent Application Ser. No. 62/672,175, filed May 16, 2018, the entirety of which is hereby incorporated herein by reference.

### BACKGROUND OF THE INVENTION

### 1. Field of the Invention

The present invention relates to dermatological treatment systems and, more specifically, to a hydro-needle system.

### 2. Description of the Related Art

Several systems have been employed to rejuvenate skin. For example, personal micro dermabrasion (PMD) systems use a rotating disk to abrade the surface of the skin so as to reduce the appearance of fine lines and wrinkles, blemishes and enlarged pores. By removing the surficial dead skin cells, new cell growth is stimulated and circulation is increased, which replaces collagen in the skin.

Micro-needling is another skin treatment that involves using a tool small needles in the tip which are driven into the surface of the skin, usually to a depth of about 0.5 millimeters. Micro-needling creates micro-punctures from the needles the skin. In response thereto, the body sends fibroblasts to create more collagen in the affected area. The punctures can also facilitate absorption of emollients into the skin surface. However, the needles of existing micro-needling systems tend to push the skin surface away from the needles as the needles are being driven, which gives rise to uneven results.

Recently, hydro-needling has been used in skin treatment. Like micro-needling, hydro-needling employs small needles to create micro-punctures in the skin. However, the needles typically include small grooves that carry a liquid from a vial into the skin. Current hydro-needling systems lend themselves to inconsistent application and can result in uneven application of the liquid, which results in sub-optimal treatment.

Therefore, there is a need for a hydro-needling system that applies needles to the skin in an even and consistent manner.

### SUMMARY OF THE INVENTION

The disadvantages of the prior art are overcome by the present invention which, in one aspect, is a hydro needle system for rejuvenating skin that includes a vial for holding a liquid therein. A needle assembly includes an array of needles extending outwardly from the vial. The array of needles has an extended position and a retracted position. The needle assembly includes an outward flat surface for placement against the skin. Each needle in the array of needles is in fluid communication with the vial so as to be configured to transport the liquid from the vial to the skin when in the extended position. An actuator is in communication with the array of needles and is configured to apply reciprocating linear motion to the array of needles so as to repeatedly cause the array of needles to reciprocate between the extended position and the retracted position. A casing holds the vial, the needle assembly and the actuator. The casing includes a skin contacting surface that supports the array of needles in a predetermined angular relationship with respect to the skin. At least a portion of the casing is transparent so that the liquid in the vial can be seen therethrough to determine how much liquid remains in the vial during use.

In another aspect, the invention is an improvement to a device for holding a hydro needle system having a transparent vial for holding a liquid therein and a needle assembly including an array of needles extending outwardly from the vial, the array of needles having an extended position and a retracted position, the needle assembly including an outward flat surface for placement against the skin, each needle in the array of needles in fluid communication with the vial so as to be configured to transport the liquid from the vial to the skin when in the extended position. The improvement includes an actuator in communication with the array of needles that is configured to apply reciprocating linear motion to the array of needles so as to repeatedly cause the array of needles to reciprocate between the extended position and the retracted position. A casing holds the vial, the needle assembly and the actuator. The casing includes a skin contacting surface that supports the array of needles in a predetermined angular relationship with respect to the skin. At least a portion of the casing is transparent so that the liquid in the vial can be seen therethrough to determine how much liquid remains in the vial during use. A holder is disposed within the casing and holds the vial and the needle assembly within a fixed position.

In yet another aspect, the invention is a hydro needling system for rejuvenating skin that includes a hydro needle unit, including: a transparent vial for holding a liquid therein and a needle assembly including an array of needles extending outwardly from the vial, the array of needles having an extended position and a retracted position, the needle assembly including an outward flat surface for placement against the skin, each needle in the array of needles in fluid communication with the vial so as to be configured to transport the liquid from the vial to the skin when in the extended position. An actuator is in communication with the array of needles and configured to apply reciprocating linear motion to the array of needles so as to repeatedly cause the array of needles to reciprocate between the extended position and the retracted position. The actuator includes a motor that generates rotational motion; a translator, in communication with the motor, that translates the rotational motion into reciprocating motion, the translator including: a rotating cam, coupled to the motor, that rotates as a result of the rotational motion generated by the motor; and a non-rotating cam, disposed adjacently to the rotating cam, that is forced to move outwardly from the rotating cam when the rotating cam is in a first rotational position and that is allowed to move inwardly toward the cam when the rotating cam is in a second rotational position that is different from the first rotational position; and a member that applies the reciprocating motion from the translator to the vial. A casing holds the vial, the needle assembly and the actuator. The casing includes a skin contacting surface that supports the array of needles in a predetermined angular relationship with respect to the skin. At least a portion of the casing is transparent so that the liquid in the vial can be seen therethrough to determine how much liquid remains in the vial during use. A holder is disposed within the casing and holds the vial and the needle assembly within the casing.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1A is a schematic diagram of one embodiment of a hydro needle system with the needles retracted.

FIG. 1B is a schematic diagram of one embodiment of a hydro needle system with the needles extended.

FIG. 2A is an exploded schematic diagram of one representative commercial embodiment of a hydro needle system.

FIGS. 2B-2F are orthogonal schematic diagrams showing individual parts used in the embodiment of FIG. 2A.

### DETAILED DESCRIPTION OF THE INVENTION

A preferred embodiment of the invention is now described in detail. Referring to the drawings, like numbers indicate like parts throughout the views. Unless otherwise specifically indicated in the disclosure that follows, the drawings are not necessarily drawn to scale. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

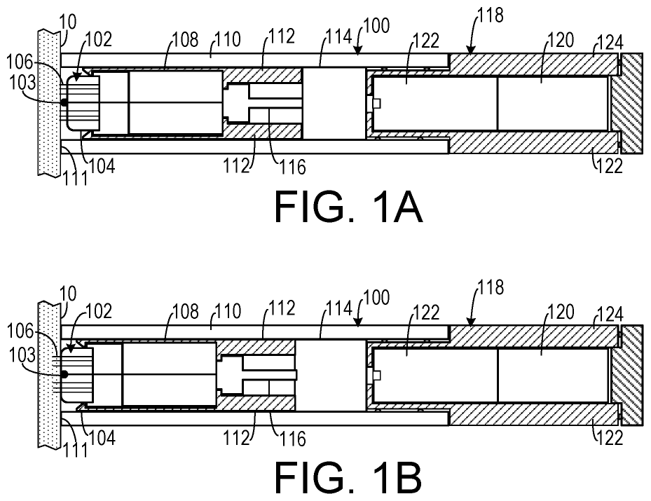

As shown in FIGS. 1A and 1B, one embodiment of a hydro needle system 100 includes a micro needle assembly 102, which includes a micro needle unit 104 that holds a plurality of needles 106 and that is coupled to a liquid-holding vial 108. The combination of the micro needle assembly 102 and the vial 108 can be referred to as a hydro needle unit 101. Certain manual hydro needle units 101 include springs 105 between the micro needle assembly 102 and the vial 108 to facilitate reciprocating application of the needles 106.

Liquid (such as a serum or other hydrating liquid) from the vial 108 flows around the needles 106 and into the user's skin 10 when a valve 103 is pushed against the skin 10. A micro needle holder 112 holds the micro needle assembly 102 and a driver member 116 (such as a cam or a piston) is configured to impart lateral reciprocating motion to the micro needle assembly 102 so as to push the needles 106 into the user's skin 10. A motor unit 118 includes a motor 122 that generates rotational force that is converted to lateral reciprocating force by translator, such as a cam 114 (such as a cam made from Rulon) or any other one of the many devices known in the art for translating rotational motion into reciprocating motion (such as a swash plate, a precision ball bearing, etc.). An electrical/electronics unit 120 supplies power to and controls the motor 122. A motor unit holder 124 holds the elements of the actuator motor unit 118.

A clear, or at least translucent, tube 110 holds the micro needle assembly 102, the driver member 116, the cam 114 and the motor unit 118. Use of a clear tube 110 allows the user to determine the amount of fluid left in the vial 108 during use. The tube 110 allows the user to see into the vial 108 to determine how much liquid is contained therein. The tube allows the user to hold the entire system 100 comfortably and has a skin contacting front surface 111 that ensures that the needles 106 enter the skin 10 at a consistent angle so that they provide even application of the liquid to the skin 10. In one embodiment, the tube 110 is made of acrylic and in another embodiment it can be made of glass. (However other materials, such as Poly(methyl methacrylate), for example, or other polymers could be used without departing from the scope of the invention.)

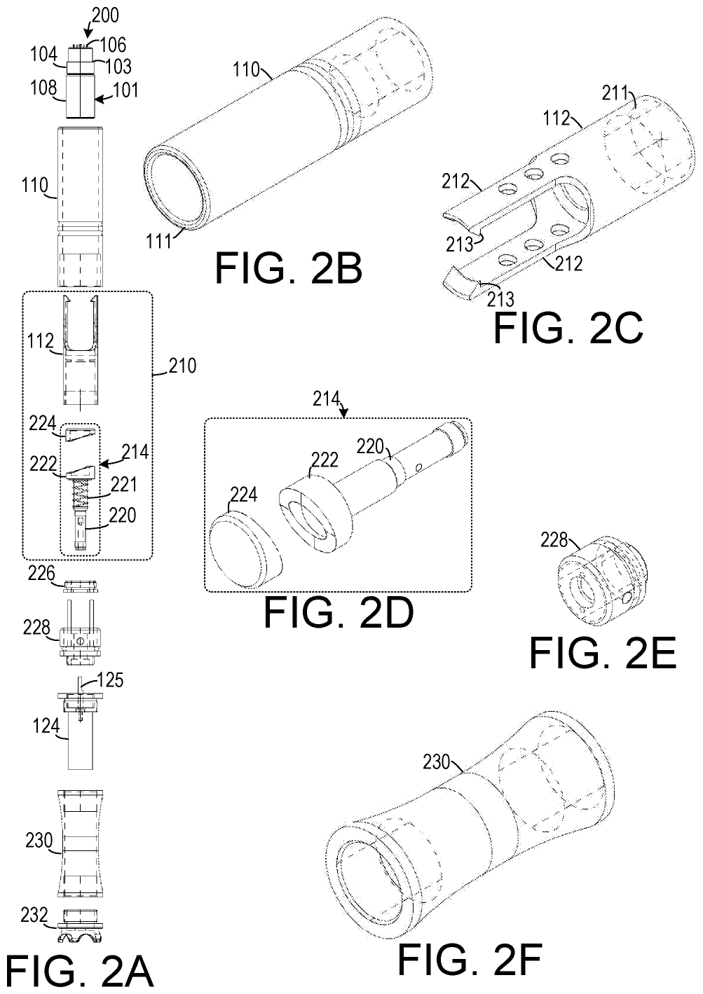

One exemplary commercial embodiment of a hydro needle system 200 is shown in FIGS. 2A-2F. This embodiment employs a hydro needle unit 101, such as a “Hydra 20,” which can be purchased from Ekai Technology, 3/F, Building E, No. 81 Zijing Road, Liwan District, Guangzhou, China. The casing 110 can include a transparent acrylic tube. The micro needle holder 112 has an external shape that is complimentary to the interior of the casing 110 and slide therein. A movement assembly 210 includes the holder 112 and a translator 214, which imparts the reciprocal linear motion on the hydro needle unit 101. The holder 112 has two arms 212 that each end in detents 213 that engage a ledge 103 to hold the hydro needle unit 101 securely. The holder 112 also defines an opening 211 through which a portion of the translator 214 passes to engage the hydro needle unit 101.

The translator 214 includes an axle 220 that is coupled to a rotating cam 222. The rotating cam 222 engages a non-rotating cam 224, both of which fit into the opening 211 defined by the holder 112. A spring 221 maintains engagement between the rotating cam 222 and the non-rotating cam 224. Rotation of the rotating cam 222 causes the non-rotating cam 224 to generate reciprocating linear motion, which is applied to the holder 112, which applies the linear motion to the hydro needle unit 101.

A rear holder 226 engages the casing 110 (both of which can be threaded) to hold the movement assembly 210 in the casing 110 and a retainer 228 is threaded into the holder 226. A motor 124 (such as an electric motor) imparts rotational motion onto a spindle 125. The spindle 125 engages the axle 220 of the rotating cam 224, causing it to rotate. Rotation of the rotating cam 224 causes reciprocal linear motion of the non-rotating cam 224, which causes reciprocal linear motion of the holder 112, which causes insertion and extraction of the needles 106.

A handle 230 is used to hold the motor 124 and engage the casing 110. A crown 232 is attached to the handle 230 and holds the motor 124 within the handle 230.

Although specific advantages have been enumerated above, various embodiments may include some, none, or all of the enumerated advantages. Other technical advantages may become readily apparent to one of ordinary skill in the art after review of the following figures and description. It is understood that, although exemplary embodiments are illustrated in the figures and described below, the principles of the present disclosure may be implemented using any number of techniques, whether currently known or not. Modifications, additions, or omissions may be made to the systems, apparatuses, and methods described herein without departing from the scope of the invention. The components of the systems and apparatuses may be integrated or separated. The operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set. It is intended that the claims and claim elements recited below do not invoke 35 U.S.C. § 112(f) unless the words “means for” or “step for” are explicitly used in the particular claim. The above described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. A hydro needle system for rejuvenating skin, comprising:
(a) a transparent vial for holding a liquid therein;
(b) a needle assembly including an array of needles extending outwardly from the vial, the array of needles having an extended position and a retracted position, the needle assembly including an outward flat surface for placement against the skin, each needle in the array of needles in fluid communication with the vial so as to be configured to transport the liquid from the vial to the skin when in the extended position;
(c) an actuator in communication with the array of needles and configured to apply reciprocating linear motion to the array of needles so as to repeatedly cause the array of needles to reciprocate between the extended position and the retracted position; and
(d) a casing that holds the vial, the needle assembly and the actuator, the casing including a skin contacting surface that supports the array of needles in a predetermined angular relationship with respect to the skin, at least a portion of the casing being transparent so that the liquid in the vial can be seen therethrough to determine how much liquid remains in the vial during use.

2. The hydro needle system of claim 1, further comprising a holder, disposed within the casing, that holds the vial and the needle assembly.

3. The hydro needle system of claim 1, wherein the actuator comprises:
(a) a motor that generates rotational motion; and
(b) a translator, in communication with the motor, that translates the rotational motion into reciprocating motion; and
(c) a unit that applies the reciprocating motion from the translator to the vial.

4. The hydro needle system of claim 3, wherein the translator comprises:
(a) a rotating cam, coupled to the motor, that rotates as a result of the rotational motion generated by the motor; and
(b) a non-rotating cam, disposed adjacently to the rotating cam, that is forced to move outwardly from the rotating cam when the rotating cam is in a first rotational position and that is allowed to move inwardly toward the cam when the rotating cam is in a second rotational position that is different from the first rotational position.

5. The hydro needle system of claim 3, further comprising a motor unit holder that holds the motor therein, wherein the casing is removably coupled to the motor unit holder

6. The hydro needle system of claim 1, wherein the casing comprises a transparent tube.

7. The hydro needle system of claim 6, wherein the transparent tube comprises a selected one of a plastic tube and a glass tube.

8. In a device for holding a hydro needle system having a transparent vial for holding a liquid therein and a needle assembly including an array of needles extending outwardly from the vial, the array of needles having an extended position and a retracted position, the needle assembly including an outward flat surface for placement against the skin, each needle in the array of needles in fluid communication with the vial so as to be configured to transport the liquid from the vial to the skin when in the extended position, the improvement comprising:
(a) an actuator in communication with the array of needles and configured to apply reciprocating linear motion to the array of needles so as to repeatedly cause the array of needles to reciprocate between the extended position and the retracted position;
(b) a casing that holds the vial, the needle assembly and the actuator, the casing including a skin contacting surface that supports the array of needles in a predetermined angular relationship with respect to the skin, at least a portion of the casing being transparent so that the liquid in the vial can be seen therethrough to determine how much liquid remains in the vial during use; and
(c) a holder, disposed within the casing, that holds the vial and the needle assembly.

9. The improvement of claim 8, wherein the actuator comprises:
(a) a motor that generates rotational motion;
(b) a translator, in communication with the motor, that translates the rotational motion into reciprocating motion; and
(c) a member that applies the reciprocating motion from the translator to the vial.

10. The improvement of claim 9, wherein the translator comprises:
(a) a rotating cam, coupled to the motor, that rotates as a result of the rotational motion generated by the motor; and
(b) a non-rotating cam, disposed adjacently to the rotating cam, that is forced to move outwardly from the rotating cam when the rotating cam is in a first rotational position and that is allowed to move inwardly toward the cam when the rotating cam is in a second rotational position that is different from the first rotational position.

11. The improvement of claim 9, further comprising a motor unit holder that holds the motor therein, wherein the casing is removably coupled to the motor unit holder

12. The improvement of claim 8, wherein the casing comprises a transparent tube.

13. The improvement of claim 12, wherein the transparent tube comprises a selected one of a plastic tube and a glass tube.

14. A hydro needling system for rejuvenating skin, comprising:
(a) a hydro needle unit, including: a transparent vial for holding a liquid therein and a needle assembly including an array of needles extending outwardly from the vial, the array of needles having an extended position and a retracted position, the needle assembly including an outward flat surface for placement against the skin, each needle in the array of needles in fluid communication with the vial so as to be configured to transport the liquid from the vial to the skin when in the extended position;
(b) an actuator in communication with the array of needles and configured to apply reciprocating linear motion to the array of needles so as to repeatedly cause the array of needles to reciprocate between the extended position and the retracted position, the actuator including:
(i) a motor that generates rotational motion;
(ii) a translator, in communication with the motor, that translates the rotational motion into reciprocating motion, the translator including: a rotating cam, coupled to the motor, that rotates as a result of the rotational motion generated by the motor; and a non-rotating cam, disposed adjacently to the rotating cam, that is forced to move outwardly from the rotating cam when the rotating cam is in a first rotational position and that is allowed to move inwardly toward the cam when the rotating cam is in a second rotational position that is different from the first rotational position; and
(iii) a member that applies the reciprocating motion from the translator to the vial;
(c) a casing that holds the vial, the needle assembly and the actuator, the casing including a skin contacting surface that supports the array of needles in a predetermined angular relationship with respect to the skin, at least a portion of the casing being transparent so that the liquid in the vial can be seen therethrough to determine how much liquid remains in the vial during use; and
(d) a holder, disposed within the casing, that holds the vial and the needle assembly.

15. The hydro needling system of claim 14, further comprising a motor unit holder that holds the motor therein, wherein the casing is removably coupled to the motor unit holder

16. The hydro needling system of claim 14, wherein the casing comprises a transparent tube.

17. The hydro needling system of claim 16, wherein the transparent tube comprises a selected one of a plastic tube and a glass tube.
